SEQUENCE LISTING (7 sequences)

SEQ ID NO: 1 - protein, 531 aa, Rattus norvegicus
MPKPDSEAGTAFIQTQQLHAAMADTFLEHMCRLDIDSAPITARNTGIICTIGPASRSVEMLKEMIKSGMNVARLNFSHGTHEYHAETIKNVRAATESFASDPILYRPVAVALDTKGPEIRTGLIKGSGTAEVELKKGATLKITLDNAYMEKCDENILWLDYKNICKVVEVGSKIYVDDGLISLQVKEKGADYLVTEVENGGSLGSKKGVNLPGAAVDLPAVSEKDIQDLKFGVEQDVDMVFASFIRKAADVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAVFHRLLFEELARASSQSTDPLEAMAMGSVEASYKCLAAALIVLTESGRSAHQVARYRPRAPIIAVTRNPQTARQAHLYRGIFPVLCKDAVLDAWAEDVDLRVNLAMNVGKARGFFKKGDVVIVLTGWRPGSGFTNTMRVVPVP

SEQ ID NO: 2 - protein, 531 aa, Rattus norvegicus
MPKPDSEAGTAFIQTQQLHAAMADTFLEHMCRLDIDSAPITARNTGIICTIGPASRSVEMLKEMIKSGMNVARLNFSHGTHEYHAETIKNVRAATESFASDPILYRPVAVALDTKGPEIRTGLIKGSGTAEVELKKGATLKITLDNAYMEKCDENILWLDYKNICKVVEVGSKIYVDDGLISLQVKEKGADYLVTEVENGGSLGSKKGVNLPGAAVDLPAVSEKDIQDLKFGVEQDVDMVFASFIRKAADVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAIYHLQLFEELRRLAPITSDPTEAAAVGAVEASFKCCSGAIIVLTKSGRSAHQVARYRPRAPIIAVTRNPQTARQAHLYRGIFPVLCKDAVLDAWAEDVDLRVNLAMNVGKARGFFKKGDVVIVLTGWRPGSGFTNTMRVVPVP

SEQ ID NO: 3 - DNA, 11788 bp, Rattus norvegicus
cccgggcgagcgccgggagggtggagagtcaccgggcggggctggaggaatgtccgggacctataaatctgggcaacgccctggtaggccagggcagatggggcacctgggcagaattccaaaatgggattatgtagcctctgaggtcctaaagcaacaggtggcggaccacccggggatctaggggtggtggcggcggtggacccgagggcgggtcctgcctcctcaccacttccccattggccatcagaatgacccatgcgcaattttggtttgcaatgtccttccgccacggaaggtagtccccctcaaaagggcaacctgcttgtcccgcctaccctgcgactctctcagaaggtgcgggtgcctgttgagaggcggggctctgctagctgctgcccggattgggcgaggggcggggctgcggagggattgcggcggcccgcagcagtgataaccttgaggcccagtctgcgcagccccgcacagcagcgacccgtcctaagtcgacagacgtcctctttaggtattgcaacaggatctgaagtacgcccgaggtgagcggggagaacctttgccattctgtggcccagagccaatgccatagtggatcactagtgggtggctgcaatgtcaggtggcagagccaggcttgggtgcatgttctttaggtatccagaaaagccgatgtccagggtagagtgcgggtctttgggaaggcagggaagagcaaggagatgtggagtgcaggctgtgtgctcaggagtttgaaaggagcctcgtggcctgaaaattgattcaagtctctggtattctgtgaggtacatcccaggttctcgcttactcagccaactatccagctcagtgagtgcttagctgtaagtcgtaataaagtaaaagcaaaggtcaggacagctgttgggattgtgacctgggtgattaattgcctggaacagatgttcagactactgtgtcgtggtccctatcagacagagaaggagactctagatagccagaactggggctgggaaagctgggtctaatgtcagctcttgggaagagtagtgactttcaaggtcaacctcggttgcacagtggatttcaggccagcatagactagctactgtgtgtgattccatctcctaggttactgaagactccttggtgaggttgtaatgactttggagccatttcaatgtccttgtgcaaaatttgctttgtgtgtcttgtacatggtgctgcttttttgcttcttgtccagaaaaaaacttgttgcctgtaatttggaccttattcctgtgtcggatagaggctaaggaatacatttaggtgatacctcaaacctaggactttaaaaagaaaggacttgggacagggttcgtctcttggcagcaggatcgtttaggtcatggcgaacatgataaagaggctatgtcagtgccaaagcaggtttgacattgtcgtgataggcttcccagatgttcgtgtgctgacacattagctacgtgctctgcaggagtggggaggtttccttgtgtgggaaggtatgttctctctagggttggtgtgaagctcttcaatagatggcagccgttgtaagaagctctgcttaaactggcagccagggtgtgtatgtgcactgggaaggaagctacttttgttcaccaaggtctttgctgttcgttcatagtcacaggagccctttgcccaatttgagtagtacccacataaccagcacgtgccaggcctctgagtgacttgtgcacaaagcagtcttattgggttaatgtgtaagcgggagacagtctcctatgccaaatggttcattaagcactgcctagtctaattgaactcttcaaatctccgggatccaggatctcagaaaccatgcccaagccagacagcgaagcagggactgccttcattcagacccagcagctccatgcagccatggctgacaccttcctggaacacatgtgccgcctggacattgactccgcacccatcacggcccgcaacactgggatcatctgtaccattggtgagtgagtgtggcccccttcgcagggcttctgcctggtttgaaaggcgtaataaccattgcagggctaacctaggttctgagagacatgtccacacttttagaggaacgtattggtgtacttcttctgcacttggtgaggttcagggtggtctagtggcctttgcaggagtagttcagggacttcagatctttttcctgtgctgtcactccactttctgcagaatttatagaaaatgatagtgtccttccttacatataaattaagagagtccttccttacatataaatcaagagagttgactagcacagtagtctttctggactttggaaagacctttctctaaatttgtggaggtattaaaaacaaaggacaaggagttgcctttgatagtagcagatttcatatactttcctacctgagaatcatgattttcttcctgtggctctaaatgtttcttggtaggccctgcttcccgatctgtggagatgctgaaggagatgattaagtctgggatgaatgtggctcggctgaatttctctcatggaacccatgaggtgagtggcagcttgatccaggaggttcgggacttgctgctgctgtgctccaggcttctcagtgaatggacgttgctctacttacatttagatcccctctctccctcttcccctctggccaacttcattggcatcaacaacctgcctgctttgtcgccggccaggttatccctaccatcaagcttcaccagggctttcttattctatgtgcaccttcgatgaggggctgtggctgtctgtcttggatgaggggctgtggctgtctgtcttggttttaaccattcctggagacggtggaatgaatcgtagctccctcgagctcttggcctaattgatcatctttggctccagggtggctcagggcatgggaactggacttgggtgtggagaacacctacctgtcaatctccccttctttctctccagtaccatgcagagactatcaagaatgtccgtgcagccacagaaagctttgcatctgatcccattctctaccgacctgttgcggtggctctggatacaaagggacctgagatccggactggactcatcaagggcgtgagtatccaggagtttaggtctttaaatgagaatatttttcatctgcctggtaggaattattatagatgcacatttttggtatgtgaataacatacttaagtctcactctggggacctggtttgttgtttgttttgtttgtttccctcaataaacaaattcaggatttacagaaaggtgatcggtttcttggggctttgagccagagtttgagcgccgccatcagggtgttggcgtccacagtcacacgcctctgctgtctttaatctagagcggcaccgcagaggtggagctgaagaagggagccacactgaagatcaccctggacaacgcctacatggagaagtgcgacgagaacatcctgtggctggactataagaacatctgcaaggtggtggaggtgggcagcaagatctacgtggacgatgggctcatctccctgcaggtgaaggagaaaggtatgtgtggtgtacagtccacggcccaatgccactcccatccccagaactctggtaagcacttaacctagcatgtatgaattggtctcccaaggtcaaaagtttaaggtggttgttggtctgcatccctggcctgtctgaaacactgcctgagaaaaaatagacaaataacctacaaaggcctatgtgtacacctctaccctttagttccagcactcgggaatcagcaggtgtgtgagttctcatgtgtaaggactactcctgtatgcctagaatgagtctagagttctcttggcttctcacaaactgagatagatggtcttgatccctttcacacaggtgctgactacctggtgacagaagtggaaaatggtggctccttgggcagcaagaagggcgtgaacctgcctggtgctgctgtggacctccctgctgtgtcagaaaaggacatccaggacctgaagtttggggtggagcaggacgtggacatggtgtttgcgtctttcatccgcaaggcggctgacgtgcatgaggttaggaaggtcctgggagagaagggcaagaacatcaagatcatcagcaaaatcgagaaccatgaaggtgtccgcaggtgaaggtccactcctaccgtgtgcctggggtgtggccttgagggcacctctgtcagggcccaggaaagctctgtccattctttggttgtacttcctgttctatagcatctttctttttatcaggtttgatgagattttggaggccagcgatggaatcatggtagctcgtggtgacctgggcattgagattccggcagagaaggtcttcctagctcagaagatgatgattggacgatgcaaccgagctgggaagccagtcatctgcgccacccaggcatgtgctatcccttccttctgtgttctccacctaggagacctggtcttgacctggcctttaggtacacgtacccgcacatagctatgacctgcgtacctgtgcaagcttcggggaattgccctggaatcatcactgaagatgtcctgctcttccattatttagtgactttcatttagcggtggtctcttacttaataaaaacccttgtttgtcccctcctagatgctggagagcatgatcaagaagccacgccccacccgtgctgaaggcagtgacgtggccaatgcagtcctagatggagctgactgcatcatgctgtccggagaaacagccaaaggggactaccctctggaggctgttcgcatgcagcacctggtgagtaagtcctcagagtcctggggtagaagagctctgctggagaggcctctgtccagtcttgttacattgctcccgtcacagcaaggagagtgaggtttgtggagtgtgcttgagtttcattgtgctttcactgcctgcacctgcccctttgtcctgctctggggattacataggccacatctggctaaaatatcaaggtcctaggatgcagtcaagggatgccttccttgtggacacccagagggcctgggtacctctattctaaaggagccaagagtttgttcagctattcgccttgttacctctcatttggtctcctgtggtctgcccatgggcaatgcttctctccactgcagctgtagccatactgagctgctttaagctggccgtgcatggtgcctgtgacatgggacttcctcccttgctgtgccagacccaactcggggctacaaatagctctgggggtggggaatgggtgctaatttagcaggttctgtctggtctaggaattataaagacttctcaggcatattatgtgctcttttgattaagtcttctggtttcagtaagatagggtctggcgcaagcttgtaattccaacaatcaaatcaagcctgcggcaggaagatggcttgagattaggctggactagaatgagacatcttgtagaagcaacaggcgtcagtgacctgtccctgcggactttccacccaggcctccgtcttctgtgttctgctccagaacttcttccggagcagaacaattatctggggcatcagttagggaccgttgcctgataaaggcatggtggattagctttttgggggttttctattcccttttcagccaacaaagaaccacagtatcttttgtggctccagtttcccacaagcccccccaaacttaagacaagtgaaagaaaagaaggaagccccttctgggttaggcatcttaccttgctttatatgtatatgagagttgagtagattccatgatctttccatggatttatctgaattatagctgtagccagatgtttgcctatattgaaacagaccagcgctttgactcggctggtcatgacctgttctgattggaattgtgactggtcctcatagcttctcatagctgcaggcattctctcccagaagacttttcccttccttcaatctcccccacagtagctgccttcctggtttacaccaaaaccccttttctacttacctggcctcacagtggtcaggaacaggactttgaccaggtattctaaagcatgtagtcacataaatgtatttttggatagctcaagaagaccttgcccaaagtggcggttgatgagattaattacaattaattatatgtgtatgagttagttttggtgtcctgtccttttctcatgccctggtctttcctatgaacagacaccttagaacctcgaggctgggattgcatggccctgctcagaagatgagtcacagagtccgggttagactgtggctaccccctcagggatacaaatttctctatcagttaacacttaggacagcttctcccctttctttatctgtttgctgtttcctcctgtgtctaactgattcagttcaaaacctgtaattaaaagctagacatcccagctgtggttgcttcctgtccatgcctcttgtcctttgggcttgcctgcattccatgcttaaccaaaagattactttccacttgcaaatctgctaatgctaccaataaaatcgagtgctggtttcatatcattcctggaattgcactctctaaaactttatttctttatttctaatgcttggttttacttgaacaggtggtctgtatataacacatttgctatcctgtaaccgttttaattattgcagtttgaatctgtgtcttgaaaggcctgtgtgctttcccaggcgtctgcctcctcacatggctgttcagtgtaccgtgttcaagtgagccagtcgaccactgttctgtttagaacttgtgcactgcaactttggctcttttgaccctcacccccagctttcagagctgcccgagtgtttccactgtaagccaagtgcaagcgctcactcttgtgtgtaggcggagttggatgccttgtaaccacatagccatgtgaggaggggacgcctttttcttcctgtaagctgtgtcaggaggcagtgtggtcaagcggaagtgtagttggctccaccttggcatctttccatgccagggtccctttctcagctttaatcaaaaacaaaagaaatgatggatggtgcatgcctagcacttgggaggctgaggcaaagaaaatagtggtgttttgaggctgaccatggcaagttcaaggccagtaggtgctatggtaagatcctatctcaaacctgtgtggggttgagaattggctttttttgtttgtttgtttttagaaacaggaaatcccaaaaggatactatctttccagaagggaaggaaggatctgtccgtgtgtttgaacagaaaagagagccacccaaaatcccacatataataccattccaggctttgaccatcctgcctctgtatctgcatggagaagaaaagattaacctaagagttccttcctctcattagttccctgtctttccatgtgttgtctcttgtttttgccttcatccttcttccttatccttcctaccctaaaccttacagatagctcgagaggctgaggcagccgtgttccaccgcctgctgtttgaagagcttgcgcgagcctccagtcaatccacagaccccctggaggccatggccatgggcagcgtggaggcctcttataaatgtttagcagcagctttgatagttctgacggagtccggcaggtagggccctgagggcaggtatcattataagataaccagcttctcacacaactagggcccactgtgtgcctgagcctgggcatagcctctctcctgcaggaaggcagccaaggaggtcacgatagggcaggaccaaaggattccctagtgggtacagtggaagtcacaggcactggttcaggatggttccctgtggagtttctaatcttgctcaagtttcagaaacgtgttagtgaactcatctttctcctggctttttgtgcccaggacatgttccttcccagttgcctgtgactcttctccttcatttgtgacaaagctctgacaaagccctgtcccccttcctcgtccctctggacggatgttgctcccctagattgcccgagaggcagaggctgccatctaccacttgcagttattcgaggaactccgccgcctggcgcccattaccagcgaccccacagaagctgccgccgtgggtgccgtggaggcctccttcaagtgctgcagtggggccattatcgtgctcaccaagtctggcaggtaggaggcggcagtggctccctggggatcccagagcaactctgggctgattttaagacccctggctgccatcaaaggactccaggaagcactcccaggtacatcagattaggtggctccagtgccagtcagtgcaggcctgcctcagggcctgaagcatatatacagttttgcttagttacggtgtggaagctgggcatgggtacatgcctttaatcctcccagcattcgggcagaagaggtgggcaaatgtctgcattcgtggacagtgctggtctacaaagcaagttccaggacagagccctagctcacaaaaattaactcagctggtaaatactacttgctacaccaagttagacggatggaggagggagaaaggtaacttagcaagcgtgcccaaggacatatacagtaacactttttctcatcccacttggagggagctcaatggataaagtgttggcaatctggatttggatctggcacctgtgggtttttgttgttgtttgggtttttgtttttttttttttttttaagtcacctggagaagtacgttaggcctttaggagcatggggttttggtttttaagatttatttattatatatgaactacactctccctatcttcagtcgcaccagaagagggtgattacagatgattgtgtaagccaccatgtggttgctgggagttgaactcaggacctctggaagagcagttagtgctcttaaccactgagccatctctccagccagcactgttctttcagaagcccaacacatacatgacagctcacactatctaactgcagtcctggggggaatctaatgccctcttctaacttccagccgtacatgtttatatgatgcatgtggtcaggatacccagacagctggagggaaaggaaggtgcccaacacacctgtgtgcctgtaatcccagcactcatggtacagaggtagaaagatcacaagataggtctaatctgagcaaattctgcaacagcctagatacagaaagccaaacacataaaaatcagtgtgggctgaggatgttgacctttgtgtgaattggccatgaacattgtatgcagctggaagcaagggatggggtgctgaaaaatggggaccttaaaactaattttctgaagtcctgaagtttcgagagctccaagatacaagttctatgctaatcctggactttctctgaagagttcctggttccttcaaagcccatgtaggccagctagaagcagggtattttctgcctgtggaatggccacatcccctaataccttgtgtgtcctcaagatgcctagttaattcagtaagcctgaaacagatctgatagaagtaaacatatcatttgctatcggttgaggagagaggggctccctacattcccaaaggatcttgattggccagatattaaccatgcctggtcacacctagattttccaaaaaaccatagaaattcagaggtttcttttagcaccaagttcagctgataagactcccaaaggagcagtatagtactctctggcaaatactgtccactccagggtctctgcagataccagaagtcaggagctaggcacatggtgcttggattgtaaaagttgctggcagctacaggatggttctggtgagattaggccagagctgcctcactgcctgatggaagggttcacagtgtgggagggatcctgccagccgtggtcctatgggactgcccacactgagatcaggacaatgagttaaaaaggacaggacaggtctgagggggtggccaggcactaaacagtaattgtaagtgggcaaacctgtggcctagaggtgaattagagggtgctcctttggctgactgaaagggtctcgtgacacaatggccattcttcccaccttctcaggagtgctcaccaagtggcccggtaccgcccaagggctcctatcattgctgtgacacgcaatccccagacagcccgccaggcccatctgtaccgtggcatcttccctgtgctgtgtaaggatgccgtactggatgcctgggctgaggacgttgatcttcgtgtgaacttggccatgaatgttggtatgtagctggaagcaagggatggggtgctgggaaatggggagcctaaaactaccatttcctgaagccctcacccaattttgggcccaaggcaaaattaattgcctcattaggctcttgtaagattaattggagctactgccctgtggggtggggcacacaagctgttgtggtctttgttcctatataaggttggatatcaagagacaaggaagcagctgaccctgaacttgggcaaggctggccactctagagtctaaactgaatggtgtccaaaggtccctgcagtctgagttttgcctcagcccttgtttaagctaggagacttgcaccaccttgtggtaaaaagaagtaactggcaacctgctctcctacctgaattagaagcaatagggcatttgttatcctgcctggttaggcttgtgtctgggctgggagaaagcaccaccttgtggtaggagagagtactggttgctcagttaggactgggtagggctttgcagttatccccaagtgttatatacctctactcaccaacctccttctcccctcaggcaaggcccgaggcttcttcaagaagggagatgtggtcattgtgctgactggatggcgccctggctctggcttcaccaacaccatgcgtgtagtgcctgtaccatgatgatcctctggagcttctcttctagcccctgtcccttcccctcccctatcctatccattaggccagcaacgttgtagtgctcactctgggccatagtgtggcgctggtgggctgggacaccaggaaaaattaatgcctctgaaacatgcaatagagcccagctatttttcatggccctacttgagccaggggtgaaggaggaatgcaggattggaaaccctctgactttatcacagaagggcagcattatctctgtgttctttgctcctgtagaaagttttccagagaattcccagccctggcctggaatcaggagacagcaagaacagaggctgggggcccagggttcccatgtagatgacttttggccctgtccctgacttgctttcccaacagctttggcctctctcctcgtgcactccactgctgtccctgcagatgttccactctccacctcgtactctgcagcgtctccaggcctgttgctatagtgcccacctgaatgtcaataaacagcagcggaacgacctgtgttcttttcttcctggggggcgtggttgaggctatcctctgagcagtcgaaaatgacaaccggcctaaaggctcagcctggagccgagtatataggttgcctcctccaacagcaccaagtggggctatctatgtgttggaaagctcactgttttatttcaggacagactggaaaggtctgggacaggttcagctagcacctgtaatggtgggtagaccggaggg

SEQ ID NO: 4 - protein, 606 aa, Homo sapiens [sequence omitted]

SEQ ID NO: 5 - DNA, 2466 bp, Homo sapiens
acgtgaccctgacccaataagggtggaaggctgagtccgcagagccaataacgagagtccgagaggcgacggaggcggactctgtgaggaaacaagaagagaggcccaagatggagacggcggcggctgtagcggcgtgacaggagccccatggcacctgcccagccccacctcagcccatcttgacaaaatctaaggctccatggagccaccacggggcccccctgccaatggggccgagccatcccgggcagtgggcaccgtcaaagtatacctgcccaacaagcaacgcacggtggtgactgtccgggatggcatgagtgtctacgactctctagacaaggccctgaaggtgcggggtctaaatcaggactgctgtgtggtctaccgactcatcaagggacgaaagacggtcactgcctgggacacagccattgctcccctggatggcgaggagctcattgtcgaggtccttgaagatgtcccgctgaccatgcacaattttgtacggaagaccttcttcagcctggcgttctgtgacttctgccttaagtttctgttccatggcttccgttgccaaacctgtggctacaagttccaccagcattgttcctccaaggtccccacagtctgtgttgacatgagtaccaaccgccaacagttctaccacagtgtccaggatttgtccggaggctccagacagcatgaggctccctcgaaccgccccctgaatgagttgctaaccccccagggtcccagcccccgcacccagcactgtgacccggagcacttccccttccctgccccagccaatgcccccctacagcgcatccgctccacgtccactcccaacgtccatatggtcagcaccacggcccccatggactccaacctcatccagctcactggccagagtttcagcactgatgctgccggtagtagaggaggtagtgatggaaccccccgggggagccccagcccagccagcgtgtcctcggggaggaagtccccacattccaagtcaccagcagagcagcgcgagcggaagtccttggccgatgacaagaagaaagtgaagaacctggggtaccgggactcaggctattactgggaggtaccacccagtgaggtgcagctgctgaagaggatcgggacgggctcgtttggcaccgtgtttcgagggcggtggcatggcgatgtggccgtgaaggtgctcaaggtgtcccagcccacagctgagcaggcccaggctttcaagaatgagatgcaggtgctcaggaagacgcgacatgtcaacatcttgctgtttatgggcttcatgacccggccgggatttgccatcatcacacagtggtgtgagggctccagcctctaccatcacctgcatgtggccgacacacgcttcgacatggtccagctcatcgacgtggcccggcagactgcccagggcatggactacctccatgccaagaacatcatccaccgagatctcaagtctaacaacatcttcctacatgaggggctcacggtgaagatcggtgactttggcttggccacagtgaagactcgatggagcggggcccagcccttggagcagccctcaggatctgtgctgtggatggcagctgaggtgatccgtatgcaggacccgaacccctacagcttccagtcagacgtctatgcctacggggttgtgctctacgagcttatgactggctcactgccttacagccacattggctgccgtgaccagattatctttatggtgggccgtggctatctgtccccggacctcagcaaaatctccagcaactgccccaaggccatgcggcgcctgctgtctgactgcctcaagttccagcgggaggagcggcccctcttcccccagatcctggccacaattgagctgctgcaacggtcactccccaagattgagcggagtgcctcggaaccctccttgcaccgcacccaggccgatgagttgcctgcctgcctactcagcgcagcccgccttgtgccttaggccccgcccaagccaccagggagccaatctcagccctccacgccaaggagccttgcccaccagccaatcaatgttcgtctctgccctgatgctgcctcaggatcccccattccccaccctgggagatgagggggtccccatgtgcttttccagttcttctggaattgggggacccccgccaaagactgagccccctgtctcctccatcatttggtttcctctttggctttggggatacttctaaattttgggagctcctccatctccaatggctgggatttgtggcagggattccactcagaacctctctggaatttgtgcctgatgtgccttccactggattttggggttcccagcaccccatgtggattttgggggtcccttttgtgtctcccccgccattcaaggactcctctctttcttcaccaagaagcacagaattc

SEQ ID NO: 6 - DNA, 39 bp, artificial
  misc_feature (1)..(39): hamerhead binding to a-raf partial sequece
gugggcuggcugaugagucgugagacgaaacaccuugag

SEQ ID NO: 7 - DNA, 20 bp, artificial
  misc_feature (1)..(20): a-raf partal sequece
cucaaggugucccagcccac

(19) United States
(12) Patent Application Publication
(10) Pub. No.: US 2004/0115631 A1
(43) Pub. Date: Jun. 17, 2004

(54) Nucleic acid coding for a bonding site of a protein kinase of the mitogenic signalling cascade of a clycolysis-catalysing enzyme

(76) Inventors: Ulf R. Rapp, Wurzburg (DE); Erich Eigenbrodt, Linden (DE)

Correspondence Address: Mark D Wieczorek, Post Office Box 70072, San Diego, CA, 92167, US

(21) Appl. No.: 10/311,527

(22) PCT Filed: Jun. 14, 2001
(86) PCT No.: PCT/DE01/02246

(30) Foreign Application Priority Data
Jun. 14, 2000 (DE) 10029131.7

(51) Int. Cl. C12Q 1/68; C07H 21/04; C12N 9/12

(52) U.S. Cl. 435/6; 435/69.1; 435/194; 435/320.1; 435/325; 536/23.2

(57) ABSTRACT

The invention relates to a nucleid acid coding for at least one partial sequence of a protein kinase of the mitogenic signalling cascade, whereby the partial sequence codes for a binding site for a glycolysis-catalysing enzyme. The invention further relates to a silent mutation of one such nucleic acid or a nucleic acid hybridising with one such nucleic acid or the silent mutation thereof.

### FIELD OF THE INVENTION

The invention relates to nucleic acids coding for protein kinases of the mitogenic signalling cascade, to interactions of such kinases with other substances in an organism, to screening methods for the identification of the interacting other substances, and to substances for the inhibition of the interactions in an organism as well as pharmaceutical preparations produced with such substances.

### BACKGROUND OF THE INVENTION

The role of c-raf-1 in the classic mitogenic MAP kinase cascade is well researched. Less well known are the functions and correlations of the two other rat isoforms B-raf and A-raf. raf protoonco-genes are highly conserved genes coding for serine/threonine-specific kinases of the cytoplasm. These kinases have functions in the mitogenic signal transduction. This cascade transfers signals from receptor tyrosine kinases via ras, raf, MEK and ERK to targets in the cytoplasm and basically to the regulation of the proliferation and differentiation of the cells. For details, reference is made, as an example only, to U. R. Rapp in the Oncogene Handbook, T. Curran et al., Eds., Elsevier Science Publishers, The Netherlands, 1988, pages 115-154. Whereas c-raf-1 in the human organisms is practically ubiquitous, A-raf exists in a natural form substantially in tissues of the urogenital system, and B-raf mainly in the cerebrum and testis. For further information and literature about this, reference is made to the document U.S. Pat. No. 5,869,308.

Classic biochemical examinations of tumours have proven a longer time ago already that tumours are subject to a modified metabolism. Even if this modified metabolism is not the fundamental reason for tumour diseases, it has nevertheless a considerable influence on the behaviour of a three-dimensional tumour tissue. Typically such tumour tissue has a pronounced hypoxia, i.e. lack of oxygen, due to the disorganised growth of blood vessels in the tumour tissue. This shows that an adaptation to hypoxic conditions is an important step in the tumour growth. The anaerobic consumption of glucose for the purpose of energy generation by glycolysis is thus a common feature of most tumour tissue aggregates. With regard to general, more detailed literature, reference is made to C. V. Dang et al., TIBS 24:68-72, 1999. Pyruvate kinases (PK) are key enzymes of the glycolysis and catalyse the trans-formation from phosphoenolpyruvate into pyruvate, the last reaction in the reaction sequence of the transformation of glucose into pyruvate, and play therefore an important role in the generation of ATP from ADP. Four tissue-specific isoforms are known, PK type L, R, M1 and M2 (see F. Eigenbrodt et al., Critical reviews in Oncogenesis, vol. 3, M. Perucho, Ed., CRC-Press, Boca Raton, Fla., pages 91-115, 1992). M2-PK is the embryonic form and re-places all other forms in proliferating cells and tumour cells (see G. E. J. Staal et al., Biochemical and Molecular Aspects of Selected Cancers, T. G. Pretlow et al., Eds., Academic Press Inc., San Diego, 1, pages 313-337, 1991, and U. Brinck et al., Virchows Archiv 424, pages 177-185, 1994). The M2-PK protein of the rat consists of 530 amino acids and differs from human M2-PK by a remainder only (see T. Noguchi et al., J. Biol. Chem., 261, pages 13807-13812, 1986, and K. Tani et al., Gene, 73, pages 509-516, 1988). M2-PK is a glycolytic enzyme existing in an active tetrameric and an inactive dimeric form. The transit between the two forms finally regulates the glycolytic consumption in tumour cells (see W. Zwerschke et al., Proc. Natl. Acad. Sci. USA, 96, pages 1291-1296, 1999, and S. Mazurek et al., J. Bioeneg. Biomembr., 29, pages 315v-330, 1997). The activity of M2-PK thus controls the transit of the glycolytic path and determines the relative content of glucose channelled into the synthesis process or used for glycolytic energy generation. The over-expression of M2-PK permits cells to survive under the conditions of a low oxygen level, since oxidative phosphorylation is not required for the production of ATP by PK. Generally, in malignant tumours and in the blood of tumour patients, an increased amount of M2-PK is found.

A binding of A-raf to H-ras, MEK and CK2β is known (see A. B. Vojtek et al., Cell, 74, pages 205-214, 1993, and X. Wu et al., J. Biol. Chem., 271, pages 3265-3271, 1996, and B. Boldyreff et al., FEBS Lett., 403, pages 197-199, 1997, and C. Hagemann et al., FEBS Lett., 403, pages 200-202, 1997). Equally known is a binding of B-raf and c-raf-1 to members of the 14-3-3 family (see B. Yamamori et al., J, Biol. Chem., 270, pages 11723-11726, 1995, and C. Papin et al., Oncogene, 12, pages 2213-2221, 1996, and E. Freed et al., Science, 265, pages 1713-1716, 1994, and K. Irie et al., Science, 265, pages 1716-1719, 1994). It has also been assumed that the 14-3-3 proteins equally bind to A-raf, since the binding sites are conserved in all raf-isoforms (see G. Daum et al., Trends Biochem. Sci., 19, pages 474-480, 1994). A direct binding of A-raf or other elements of the mitogenic signalling cascade to factors of the metabolic regulation is however not known.

Technical Object.

The invention is based on the technical object to find an approach for the inhibition of the anaerobic metabolism in tumour cells, and then to find effective substances at least slowing the growth of tumour cell aggregates down or promoting apoptosis in such tumour cell aggregates because of insufficient energy generation in the tumour cells.

The Findings the Invention is Based On.

By means of a two-hybrid system and by using A-raf as a bait, a PC12 cDNA library has been screened. Among others, M2-PK was found as a binding partner for A-raf. By further experiments, it has been found that the co-operation between A-raf and M2-PK leads to a displacement of the balance between the dimeric and the tetrameric M2-PK forms in favour of the tetrameric, i.e. active form. Thus A-raf is part of the glycolytic enzyme complex. Transformation of NIH 3T3 cells by A-raf will lead to an increase of the phosphoserine content of the M2-PK protein in conjunction with the displacement of the mentioned balance.

Thereby, it has been demonstrated for the first time that an oncogene of the mitogenic signalling cascade further directly acts on an enzyme catalysing the (anaerobic) glucoslysis, and this in an amplifying and tumour growth promoting manner. Such a synergism is surprising, and it can be expected that by inhibitors of the interaction, several positive effects simultaneously can be achieved for a tumour therapy.

Interactions of oncoproteins having completely different physiological mechanisms with M2-PK are known in the art, for instance pp60^{v-src} kinase or HPV 16 oncoprotein E7 which lead to a displacement of the tetrameric/dimeric ratio. These have however as a consequence a displacement in the direction towards dimeric and thus reducing activity, in contrast to A-raf.

Specification of the Invention.

The invention firstly relates to a nucleic acid coding for at least one partial sequence of a protein kinase of the mitogenic signalling cascade, the partial sequence coding for a binding site for an enzyme catalysing the glycolysis, or a silent mutation of one such nucleic acid or a nucleic acid hybridising with one such nucleic acid or the silent mutation thereof. The term nucleic acid comprises DNA, RNA and PNA. Also included in this term are double-stranded nucleic acids as well as single-stranded nucleic acids and thus also complementary nucleic acids. Silent mutations are variants in the sequence not leading to a functional difference related to the binding site for an enzyme catalysing the glycolysis, the variant with regard to the natural not mutated sequence. Silent mutations may be alleles or artificial mutations. Derivatives also fall under the invention. Derivatives are non-natural chemical modifications.

By means of such nucleic acids, it can for instance be searched for co-operation partners of the protein kinase of the mitogenic signalling cascade in the group of the enzymes catalysing the glycolysis. Further, it can also be searched for inhibitors for the binding sites. Nucleic acids according to the invention are thus at last useful in particular as a screening tool.

Preferred is a nucleic acid coding for a protein or a peptide containing the sequence A-raf (587-606) or a silent mutation of one such nucleic acid or a nucleic acid hybridising with one such nucleic acid or a silent mutation thereof. Preferably, for the purpose of the invention, this is human A-raf. For research purposes, it may however also be A-raf from non-human mammals. The sequence 587-606, in particular 602-603, was identified as a region wherein the binding site for enzymes catalysing the glycolysis is located. A nucleic acid according to the invention may be shortened relative to the full sequence of A-raf, and this in particular at the n-terminal end. It will then code for a protein or peptide consisting of the sequence A-raf (255 to 587-606) or a silent mutation of one such nucleic acid or a nucleic acid hybridising with one such nucleic acid or the silent mutation thereof. In other words, the sequence may be in a region which is limited by the sequences (255-606) and (587-606)

Furthermore, the invention teaches a cDNA of the above structure and an isolated recombinant vector containing a nucleic acid of the above structure or an expression plasmid with this nucleic acid. For the purpose of stable expression, a DNA fragment coding for a suitable viral protein, for instance gag, may also be used herein (fusion gag with for instance A-raf or A-raf fragment). By means of the expression plasmid, a transformant can be formed which in turn can be used for the production of the protein or peptide coded by the nucleic acid. For this purpose, the transformant is cultivated in a suitable manner according to conventional methods.

Another aspect of the invention is an antisense nucleic acid or ribozyme binding to a for instance oncogenic nucleic acid, in particular RNA, coding for a protein kinase of the mitogenic signalling cascade. By means of such a substance, the expression of for instance A-raf can be suppressed with the consequence of an inhibition of the (anaerobic) glycolysis in the tumour tissue, In principle, the same can be achieved with a substance having a binding site for a protein or peptide coded by a nucleic acid according to the invention, selected from the group consisting of a) de-activated enzymes catalysing the glycolysis, b) inactive proteins or peptides and c) aptamers.

In the case of a ribozyme, this may for instance be a hammerhead ribozyme attacking within the kinase domain of a raf isoform m-RNA for instance at a GTC site. The hammerhead may also attack out-of-frame ATG start codons of the kinase domain. These make a translation of an active kinase domain from the produced fragmentary mRNA extremely unlikely.

This may in particular be a kinase-inactive form of M2-PK, preferably of human M2-PK. In this case, the kinase-active form may be formed by a mutation in the region of the ADP binding site and/or the ATP binding site, in particular be selected from the group consisting of “M2-P1 K366M, R119C, T34OM, Q377K, K161M, K165M and several of these mutations”. The term kinase-inactive form comprises, also in the kinase activity with regard to normal M2-PK, reduced forms only. Inactive mutants are therefore suitable for the initiation of the apoptosis of the tumour cells, since thereby a reduction of the ATP and ADP levels is achieved. As a natural mutant, M1-PK can also be used. M1-PK is expressed in all not proliferating cells. In M2-PK expressing cells there is with an additional expression of M1-PK by competing reactions an inhibition of the cell proliferation. M1-PK and M2-PK are different splicing products, only an exon with 51 amino acids being exchanged. M1-PK and M2-PK differ in 21 amino acids only. M1-PK is not phosphorylated. Hereby, phosphorylation sites can be derived by sequence comparison with M2-PK. As a result, not phosphorylable MK-PK mutants act in a tumour sunpressing and proliferation inhibiting manner. Such mutants result immediately from the following sequence comparison, wherein potential phosphorylation sites are specified. Consequently, in particular such M2-PK mutants can be used, which are mutated at least at one of the marked sites corresponding to the sequence comparison. Alternatively or additionally, one or more mutations (in any per-mutation) may be implemented at sites of other deviations in the sequence comparison.

```
M2: ³³⁶Ile-Ala-Arg-Glu-Ala-Glu-Ala-Ala-Ile-Tyr-His-Leu-Gln-Leu-Phe-Glu-Glu
                                                                                                      P
M1: ³³⁰Ile-Ala-Arg-Glu-Ala-Glu-Ala-Ala-Val-Phe-His-Arg-Leu-Leu-Phe-Glu-Glu
M2: ³³⁷Leu-Arg-Arg-Leu-Ala-Pro-Ile-Thr-Ser-Asp-Pro-Thr-Glu-Ala-Ala-Ala-Val
                                                                                              P P
M1: ³³⁷Leu-Ala-Arg-Ala-Ser-Ser-Gln-Ser-Thr-Asp-Pro-Leu-Glu-Ala-Met-Ala-Met
M2: ⁴¹⁴Gly-Ala-Val-Glu-Ala-Ser-Phe-Lys-Cys-Cys-Ser-Gly-Ala-Ile-Ile-Val-Leu
M1: ⁴¹⁴Gly-Ser-Val-Glu-Ala-Ser-Tyr-Lys-Cys-Leu-Ala-Ala-Ala-Leu-Ile-Val-Leu
M2: ⁴³¹Thr-Lys-Ser
M1: ⁴³¹Thr-Glu-Ser
```

In the case of the aptamers, it is recommended to stabilize them against nucleic acid cracking enzymes. In the case of the proteins or peptides, these are highly specific synthetic molecules “tailored” for the binding site of the protein or peptide coded by the nucleic acid according to the invention.

Because of the glycolysis inhibiting effect of the above substances, there is also part of the invention the use thereof for blocking the co-operation or binding between a protein kinase of the mitogenic signalling cascade and an enzyme catalysing the glycolysis, in particular of the A-raf/M2-PK, and the use thereof for the production of a pharmaceutical preparation for treating cancer diseases, for instance of the urogenital system. By such a blocking, the mitogenic signalling cascade is probably simultaneously blocked, and thus a synergistic effect is obtained.

Another aspect of the invention is the use of nucleic acid according to the invention or of a protein or peptide coded thereby in a screening method for the determination of an enzyme catalysing the glycolysis and co-operating with a protein kinase of the mitogenic signalling cascade. For this purpose, first known or unknown enzymes catalysing the glycolysis are identified and if applicable isolated and characterised. These are then subjected to the experiments according to the embodiments, an interaction with one or more raf isoforms being investigated. If an interaction is detected, then this enzyme will be selected. If applicable, an inactive enzyme can be produced from the selected enzyme (inactive=smaller or no effect catalysing the glycolysis, compared to the unmodified enzyme). Further, the binding site of the enzyme catalysing the glycolysis can be determined, and a peptide or mimicry substance can be produced therefor which binds at the same site of the rat isoform. The invention finally teaches the use of a nucleic acid according to the invention or of a protein or peptide coded thereby in a screening method for the detection of a substance binding to a protein kinase of the mitogenic signalling cascade, not however catalysing the glycolysis. Herein, substances with prospective binding sites for the raf isoform binding site with an enzyme catalysing the glycolysis are subjected to a binding test, for instance according to the embodiments, and those substances which bind are selected. It is possible, subsequently or previously, to test the inactivity of the prospective substances with regard to the effect catalysing the glycolysis.

In the first-mentioned use, further binding partners can be found in healthy or sick tissue. In the last-mentioned use, inhibitors of the binding process normally taking place in healthy or sick tissue can be found.

The explanations for a claim category of the invention correspondingly apply to other claim categories. The invention finally also relates to healing processes, for instance classically by a suitable administration of pharmaceutical preparations, but also gene-therapeutically, wherein one or more substances according to claim 6 to 9 are introduced into a target cell or are produced in the target cell.

In the following, the invention will be explained in more detail, based on figures and experiments representing examples of execution only. There are:

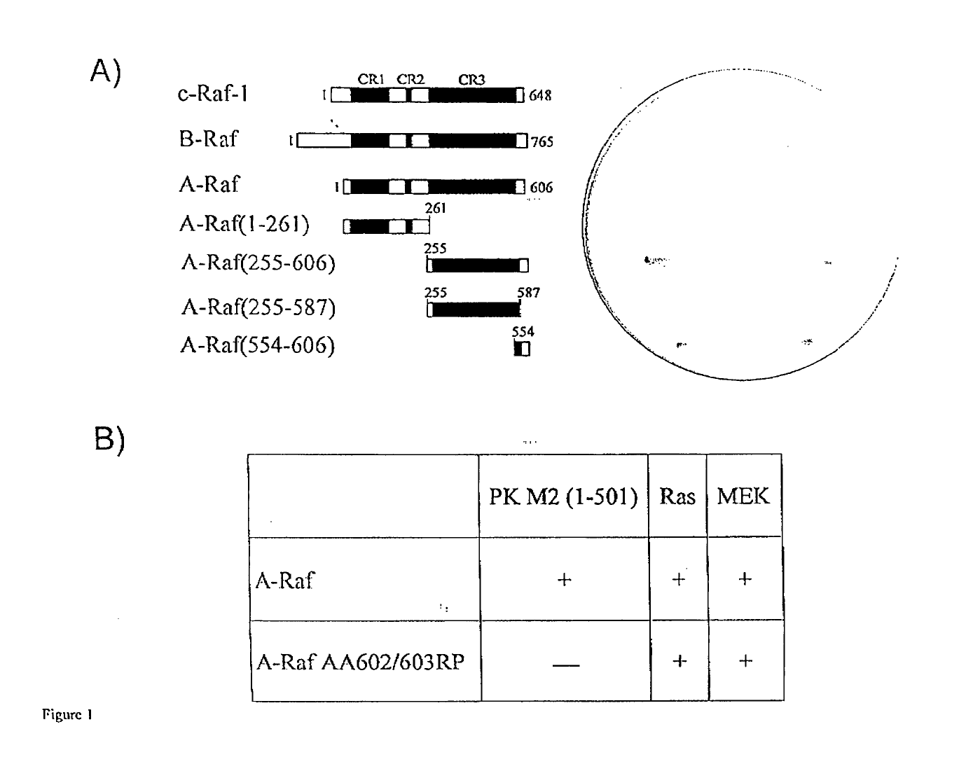

FIG. 1 the specific interaction of A-raf and M2-PK in a two-hybrid binding assay,

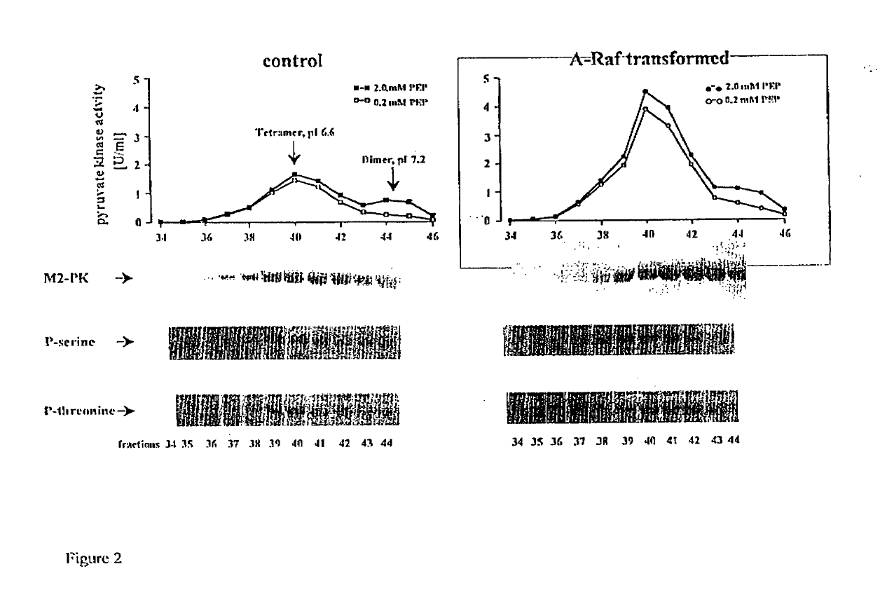

FIG. 2 the isoelectric focusing of M2-PK in control cells and in A-raf transformed NIH 3T3 cells,

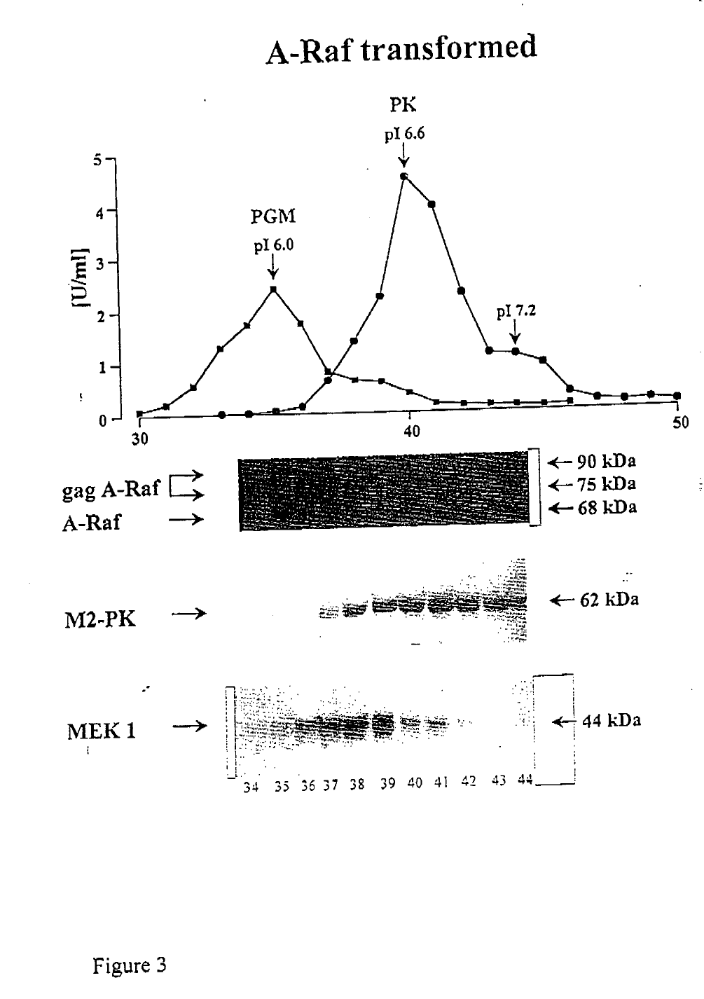

FIG. 3 the isoelectric focusing of the glycolytic enzyme complex in A-raf transformed NIH 3T3 cells,

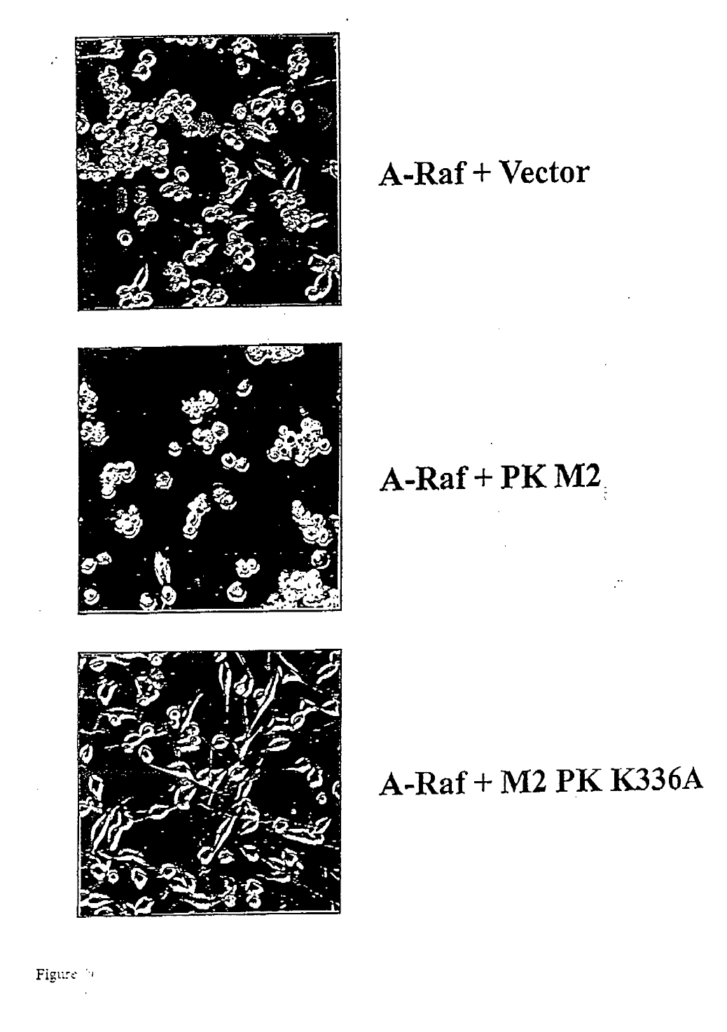

FIG. 4 the inhibition of the transformation of NIH 3T3 cells by means of kinase inactive M2-PK K336A,

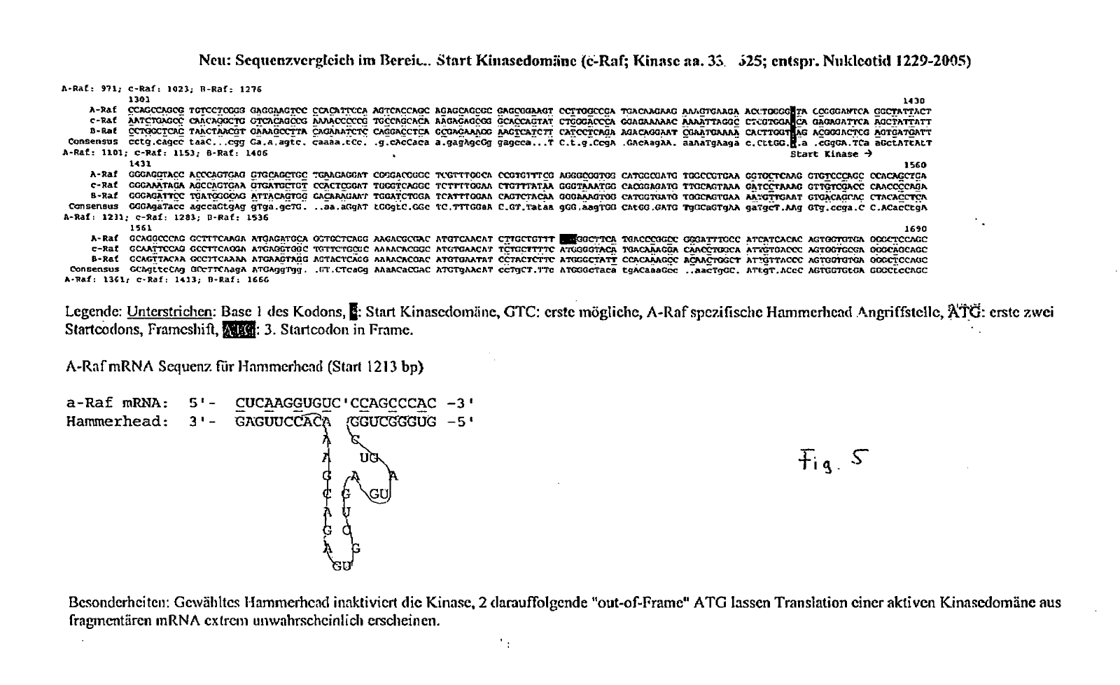

FIG. 5 sequences of raf isoforms in comparison, with marking of the start of the kinase domain, of a first GTC for a hammerhead attack, as well as of ATG start codons and representation of a hammerhead suitable for the mRNA.

Annex 1 DNA sequence of rat M1-PK and M2-PK,

Annex 2 DNA sequence of human v-raf.

1: Methods.

1.1: Plasmid Construction.

The two-hybrid vectors pPC86 and pPC97 were provided by D. Nathans (see also: P. M. Chevrey & D. Nathans, Proc. Natl. Acad. SCI, USA, 89, pages 5789-5793, 1992). Full-length A-raf, B-raf and C-raf-1 cDNA were subcloned in fusion with the Ga14 DNA binding domain in pPC97. The PC12 cDNA-library was subcloned in fusion with the Ga14 activation domain in pPC97. The cloning of the A-raf deletion constructs has been described in detail in C. Hagemann et al., FEBS Lett., 403, pages 200-202, 1997. A-raf (554-606) was amplified by using the primers 5′-CTC AAG TTG TCG ACG GAG GAG CGG CCC CTC TTC-3′ (upstream, under introduction of a SalL site) and 5′-GTG GCT TGG CGG CCG CCT AAG GCA CAA G-3′ (downstream, under introduction of a NotI site).

The HA tag and the untranslated 5′ end of the “fished” M2-PK (clone 71) were removed by that a new SalI site was produced by means of the site-directed mutagenesis kits from Stratagene, and that the resulting pPC86-M2-PK was digested, and plasmid religation. This resulted in the plasmid pPC86-PK. For the expression in E. coli and in mammal cells, M2-PK cDNA was isolated as an EcoRI fragment from pPC86 (clone 71), ligated to pGEX-2T and pcDNA3 digested with EcoRI. The untranslated region was removed from the construct by introduction of a BamHI site (mutagenesis kit Stratagene) and removal of the BamHI fragment. The 3′ end of the coding region of the M2-PK cDNA was isolated by means of PCR from the PC12 library, using the primers 5′-GCC CGG TAC CGC CCA AGG GCT C-3′ (sense) and 5′-CCA GGG CTG GGA ATT CTC TGG-3′ (antisense). Full-length M2-PK was produced by subcloning of the PCR product KpnI/EcoRI in pGEX-M2-PKΔBam and pcDNA-M2-PKΔBam, respectively, resulting in plasmids pGEX-M2-PK and pcDNA3-M2-PK.

pPC97-A-raf AA602/603RP, pcDNA3-M2-PK K366M and pGEX-2T-M2-PK K366M were produced by the mutagenesis kit from Stratagene.

1.2: Two-Hybrid Library Screening.

Yeast cultures were established at 30° C. under standard conditions in liquid or solid medium based on either YPD or minimum SD medium.

The yeast line HF7c was sequentially transformed with initially the bait plasmid and then the cDNA library. Transformants were drawn on SD medium in absence of the amino acids leucine, tryptophan and histidine. After 4 days, the growing clones were tested for the activation of the lacZ reporter gene in a β-Gal filter assay. Positive clones were further investigated by re-transformation of the isolated library plasmid, together with various bait plasmids in HF7c. Clones showing a β-Gal positive phenotype in presence of raf only were evaluated as positive and further examined by sequencing and colony hybridisation. For direct interaction tests, the yeast line HF7c was co-transformed with A-raf deletion constructs and pPC86-M2-PKΔSal.

General information about the two-hybrid vector system and variants thereto can be found in the document Biospektrum, 3/95, page 12-14, and in the literature quoted there.

1.3: Cell Cultures.

The NIH 3T3 cells were drawn under standard conditions (37° C., 5% CO₂) in DMEM (Life technologies, Inc.), supplemented with 10% heat-inactivated foetal bovine serum (hyclone), 168 mM L-glutainine (Life Technologies, Inc.) and 100 units/ml streptomycin and penicillin (Life Technologies, Inc.).

1.4: Focus Forming and Colony Yield Assay.

7×10⁴ NIH 3T3 cells and 1.5×10⁵ NIH 6A-leuk cells (stably expressing gag-A-raf, see M. Huleihel, Mol. Cell. Biol., 6, pages 2655-2662, 1986) were sown in 90 mm tissue culture dishes one day before the transfection. The transfections were performed by means of the lipofectamine method (Life Technologies, Inc.) and according to manufacturer's instructions. Focus forming was scored 10 days later. Transfected cultures were dyed with 0.4% crystal violet for the purpose of better visualisation. For the colony yield assay, cells were sown as described above and cultivated for 10 days in a selective medium containing 450 μg/ml G418 (Genetivin; GIBCO BRL; see also U. R. Rapp, Oncogene, 9, pages 3493-3498, 1994).

1.5: Isoelectric Focusing of Glycolytic Enzymes.

Control cells NIH 3T3 and A-raf transformed NIH 3T3 cells were cultivated to a cell density of 3.5×10⁶ cells/dish. For each focusing experiment, 26×10⁶ cells in 3 ml lysis buffer with a lower salt concentration (10 mM Tris, 1 mM NaF, 1 mM EDTA-Na₂ and 1 mM mercaptoethanol, pH 7.4) were extracted for the purpose of obtaining the glycolytic enzyme complex, and then centrifuged for 20 min at 40,000 g for removing solid cell substances. Homogenates were subjected to isoelectric focusing, and enzyme activities were determined in individual fractions as previously described (see S. Mazurek, J. Cell Physiol., 167, pages 238-250, 1996).

1.6: Gel Filtration Analysis of M2-PK.

Extracts of control cells NIH 3T3 and stably gag-A-raf expressing NIH 3T36A cells were produced as described in W. Zwerschke, Proc. Natl. Acad. Sci. USA, 96, pages 1291-1296, 1999.

1.7: Immunologic Detection of M2-PK, A-raf, c-raf, MEK1, Phosphoserine and Phosphothreonine.

The individual fractions of the focusing experiments or gel filtration experiments were diluted 1:10 with sample buffer. After separation on a 10% SDS polyacrylamide gel, the proteins were transferred on a nitrocellulose membrane by means of electroblotting. For the detection of the various antigens, the following antibodies were used: M2-PK: monoclonal antibodies DF4 (ScheBo Tech, Giessen, Germany); A-raf: polyclonal antibodies, which were drawn against synthetic peptides representing 12 C-terminal residues of A-raf (see C. Hagemann et al., EEBS Lett. 403, pages 200-202, 1997); gag-A-raf; goat serum produced against p30^{gag} (see M. Huleihel, Mo. Cell. Biol., 6, pages 2655-2662, 1986); c-raf: monoclonal antibodies (Dianova, Hamburg, Germany); MEK and ERK; monoclonal antibodies (Transduction laboratories); p-serine, p-threonine and p-tyrosine: monoclonal biotinylated antibodies (Sigma, Deisenhofen, Germany).

1.8; Determination of the Metabolite Concentrations.

Fructose 1,6-biphosphate, pyruvate and phosphoenolpyruvate concentrations were determined in perchloric acid extracts of the cells, as described in S. Mazurek et al., J. Biol. Chem. 272, pages 4941-4952, 1997. According to this document, the concentrations of glucose, glutamine, glutamate and lactate were also determined in the cell supernatants for the purpose of the determination of the flow rates.

2. Identification and Characterisation of the Interaction A-raf with M2-PK by Means of the Two Hybrid Binding Assay

By means of the two hybrid binding assay, 3.3×10⁷ clones of rat pheochromocytoma PC12 cDNA library were screened utilising A-raf as a bait. 73 clones showed a histidine and LacZ positive phenotype. These clones were further investigated by colony hybridisation and sequencing. They code H-ras (1 clone), MEK-2 (3 clones), 14-3-3β (5 clones), 14-3-3ζ (15 clones), 14-3-3ε/η (11 clones), 14-3-3θ (7 clones), CK2β (26 clones) and M2-PK (1 clone). Four remaining clones will have still to be investigated. With the exception of M2-PK, the above interactions are known and serve in so far as a proof for the functionality of the used system (expression and folding). The interaction detected for the first time of A-raf with an enzyme catalysing the glycolysis, namely M2-PK, is the basis for the present invention.

The isolated clone represents a partial sequence comprising a part of the untranslated region before the N-terminus and where 29 amino acids at the C-terminus are missing (M2-PK (1-501)).

In experiments with c-raf-1 and B-raf as baits, no M2-PK clones could be isolated, and this indicates an A-raf isozyme specific interaction. By means of deletion mapping, it could be shown that the interaction takes place within a very C-terminal domain of A-raf, since an interaction of M2-PK with A-raf (255-606) and A-raf (554-606) could be found, not however with A-raf (255-587). For details in this context, reference is made to FIG. 1a. The found interacting variable region is not conserved between different raf-isoforms of the mammals, which is in agreement with the observed isozyme specific binding of M2-PK in the two hybrid tests and could also provide an explanation therefor. In order to determine the exact binding domain, the mutant A-raf AA602/603RP was generated, wherein the assumed binding sites have been replaced by the corresponding c-raf-1 amino acids, and then subjected to a direct two hybrid test. In FIG. 1b can be seen that with this mutation, any interaction has disappeared, what proves that the A-raf specific binding sites are responsible for the specificity of the interaction with M2-PK.

3: Investigation of the Effect of the Interaction A-raf with M2-PK for the Metabolic Regulation.

The transition from the inactive dimeric into the active tetrameric form of M2-PK determines the glycolytic flow in tumour cells. For the purpose of the investigation of a possible effect of the interaction A-raf with M2-PK on this in vivo, isoelectric focusing experiments were performed. Two peaks could be separated for the tetrameric form (fractions 35-42) and the dimeric form (fractions 43-47), as can be seen from FIG. 2. In contrast to the dimeric form, the tetrameric form has a high affinity to its substrate phosphoenolpyruvate (PEP). In agreement with the differential substrate affinities, it was found that the tetrameric form is equally active for high (2 mM) and low (0.2 mM) PEP concentrations, whereas the dimeric form is less active at low concentrations (FIG. 2).

In FIG. 2 can be seen in detail the determination of the M2-PK activity in the presence of 2 mM and 0.2 mM PEP in the various fractions (upper portion) and the determination of M2-PK, p-serine and p-threonine by direct immunoblotting after SDS gel electrophoresis of the various fractions.

The transformation of NIH 3T3 cells by stable expression of a fusion between the A-raf kinase domain with viral gag-protein (gag-A-raf, see M. Huleihel et al., Mol. Cell. Biol., 6, pages 2655-2662, 1986) led to a selective increase of the tetrameric form (quantity of tetramers in control cells: 69%; after A-raf transformation; 76%), reference being made to FIG. 2.

FIG. 3 shows in its upper portion the determination of the activities of pyruvate kinase (circles) and phosphoglyceromutase in the various fractions. pI values for some fractions are given as references. In the lower portion, the detection of A-raf, gag-A-raf, M2-PK and MEK 1 by direct immunoblotting after SDS gel electrophoresis of the various fractions is shown. It can first be concluded that the tetrameric form of M2-PK co-focuses with enolase-3-phosphate hydrogenase (not shown) and a part of phosphoglyceromutase type B. A-raf and c-raf focus in the glycolytic enzyme complex in fractions 35-37 and gag-A-raf in fractions 40-44. A proteolytically modified form of gag-A-raf having a molecular weight between 45 and 50 kDa focused in the fractions 39-42 (not shown). MEK 1 and MEK 2, the substrates of the raf kinases, focused both in the same fractions 34-45 of the glycolytic enzyme complex. Most part of ERK 1 and ERK 2, the substrates of MEK focused outside the glycolytic enzyme complex in the fractions 32-35 (not shown) M2-PK, A-raf, gag-A-raf, c-raf, MEK and ERK were detected with the antibodies specified in the section “Methods”.

The immunologically detectable amount of M2-PK protein and the amounts of phosphoserine and phosphothreonine in the M2-PK protein were determined densitrometrically by means of the Scion Image Program (Beta 3B-Version, Scion Corporation). In comparison to control cells, the total content in M2-PK protein increased by 1.3 times in A-raf transformed cells, whereas the phosphoserine content of the M2-PK protein increased by 2.6 times and that of the phosphothreonine by 1.2 times. The ratio between phosphoserine and M2-PK protein increased from 0.7 in control cells to 1.3 in A-raf transformed cells. The ratio for phosphothreonine was however unchanged. The highest phosphorylation degree was found in the fractions 35-38, where A-raf and c-raf were also positioned. Therefrom results that the A-raf transformation will selectively increase the phosphoserine content of the M2-PK protein. The same increase in the phosphorylation degree and content in M2-PK protein was also found in other tumour cell lines, such as pp60^{v-src} transformed NIH 3T3 cells and glioma cell lines.

In another experiment the glycolytic complex was determined by extractions of the cells with high phosphate concentrations and the ratio between the tetrameric and dimeric forms of M2-PK directly by gel permeation. In agreement with the data of the isoelectric focusing, the gel permeation also showed a displacement of the dimeric form to the tetrameric form in A-raf transformed cells (tetrameric form in control cells: 77%, in A-raf transformed cells: 87%). In A-raf transformed cells, the intensity of the phosphoserine colouration of tetrameric M2-FK was stronger than in control cells.

In full agreement with the changes in the M2-PK activity, it was found that the A-raf transformation increases the glycolytic flow rate (see table 1). Simultaneously, a decrease of the fructose 1,6-biphosphate level from 63.8±13.1 (4) nmol/mg protein in control cells to 39.5±13.6 (5) nmol/mg protein (x±SD, p<0.050) was found. Pyruvate levels increased from 6.9±0.9 (4) nmol/mg protein in control cells to 9.3±2.7 (5) nmol/mg protein in A-raf cells (x±SD, p<0.054). Phosphoenolpyruvate concentrations were riot significantly changed.

Whether the metabolic changes were essential for the transformation and proliferation of the cells, was investigated in over-expression experiments, NTH 3T3 cells were transfected with gag-A-raf and M2-PK cDNA, and the focus generation after 10 days cultivation was investigated (Table 2). Whereas A-raf led to a generation of 2 foci only per 1 μg transfected DNA, the co-transfection resulted in an increase of the count to 6 foci per 1 μg DNA, which confirms a co-operative effect of A-raf and M2-PK in the cell transformation. In order to test whether a highly efficient transformation by A-raf requires M2-PK, a kinase-inactive form of M2-PK, M2-PK K366M, mutated at the assumed ADP binding site, was used, hoping to obtain an inhibiting mutant which inhibits the A-raf M2-PK co-operation or binding. This mutant has in fact the property of fully suppressing the focus generation in NIH 3T3 cells. In a colony yield assay, it was found that the M2-PK K366M mutant reduces the creation of colonies of stably A-raf expressing NIH 6A-leuk cells, so that under G418 selection only 18 colonies per 1 μg transfected DNA grew, whereas wildtype M2-PK expression resulted in a growth of 75 colonies per 1 μg DNA (Table 1). Furthermore, the growth under wildtype M2-PK was capable to promote the transformed morphology of NIH 3T3 cells, whereas M2-PK K366M does not show this, but actually antagonised the morphological transformation by A-raf. The cells namely showed a rather flat, less retractile phenotype, as can be seen in FIG. 4 (empty vector: pcDNA3) Protein expression (M2-PK and A-raf) were checked in all experiments by means of western blots (data not shown).

TABLE 1

| | Control | A-raf | | | |
|---|---|---|---|---|---|
| Metabolite | nmol/h*10⁵c. | X_{g}.DF^{±1} | | Sign. | |
| Glucose cons. | 24.0.1.2(9) | 57/5.1.2(9) | | p < | 0.01 |
| Lactate prod. | 52.5.1.0(10) | 79.4.1.0(10) | | p < | 0.001 |
| Pyruvate prod. | 1.7.1.0(10) | 4.2.1.0(10) | | p < | 0.001 |
| Glutamine cons. | 4.4.1.2(10) | 11.5.1.2(10) | | p < | 0.01 |
| | | x ± | SD | | |
| Glutamate convers. | 0.7 ± 0.02 | −1.1 ± | 0.02 | p < | 0.01 |

For the calculation of the metabolite conversion, the dependence from the cell density had to be taken into account. For the conversion of glucose, lactate, glutamine and pyruvate, a logarithmic transformation of the data was used, since the data were distorted towards right. X_{g}.DF^{±1} is the delogarithmised form of the arithmetic mean and of the standard deviation of the data logarithmically transformed before. For the statistical analysis, a one-way analysis of the covariance was performed, with the cell density as a covariable. In the case of glutamate, positive values are mean production, and negative values indicate consumption.

TABLE 2

| Metabolite | Control | | A-raf | | Sign. |
|---|---|---|---|---|---|
| Fructose 1,6-bis- | 63.8 ± | 13.1(4) | 39.5 ± | 13.6(5) | p < 0.05 |
| phosphate | | | | | |
| Pyruvate | 6.9 ± | 0.9(4) | 9.3 ± | 2.7(5) | p < 0.05 |
| Phosphoenolpyruvate | 0.3 ± | 0.3(4) | 0.9 ± | 0.8(5) | — |
| ATP/ADP | 3.1 ± | 0.9(4) | 8.3 ± | 3.1(4) | p < 0.05 |
| AMP | 7.6 ± | 2.1(4) | 1.8 ± | 1.5(4) | p < 0.01 |
| IMP | 4.2 ± | 2.3(4) | 0.4 ± | 0.2(4) | p < 0.05 |
| Inosine | 3.8 ± | 1.3(4) | 0.8 ± | 0.5(4) | p < 0.01 |

TABLE 3

| | Foci/μg | Colonies/μg DNA |
|---|---|---|
| A-raf + vector | 2 | 597 ± 30 |
| A-raf + PK-M2 | 6 | 576 ± 30 |
| A-raf + PK-M2 K366M | 2 | 156 ± 15 |

NIH 3T3 cells were transfected with A-raf, M2-PK and M2-PK K366M in the mentioned concentrations. Focus generation was determined after 10 days growth. NIH 6A-leuk cells stably expressing against gag-A-raf were transfected with M2-PK and M2-PK K366M. Colonies of surviving cells were counted after 10 days G418 selection (right-hand column). Vector=empty vector pcDNA3.

4. Sequence Comparison in the Region Start of the raf Kinase Domain and Hammerhead.

FIG. 5 shows a first GTC for an attack of the shown hammerhead at the corresponding mRNA. Of course, another target sequence is also possible for the hammerhead attack, same as other positions of identical target sequences. The hammerhead can easily be adapted by the man skilled in the art. The only thing that is essential is that the translation of an active kinase domain is reduced or suppressed.

1. A nucleic acid coding for at least one partial sequence of a protein kinase of the mitogenic signalling cascade, the partial sequence coding for a binding site for an enzyme catalysing the glycolysis, or a silent mutation of one such nucleic acid or a nucleic acid hybridising with one such nucleic acid or the silent mutation thereof.

2. A nucleic acid according to claim 1 coding for a protein or a peptide containing the sequence A-raf (587-606), in particular (602-603), or a silent mutation of one such a nucleic acid or a nucleic acid hybridising with one such nucleic acid or a silent mutation thereof.

3. A nucleic acid according to claim 1 or 2 coding for a protein or peptide consisting of the sequence A-raf (255 to 587-606) or a silent mutation of one such nucleic acid or a nucleic acid hybridising with one such nucleic acid or the silent mutation thereof.

4. A cDNA according to one of claims 1 to 3.

5. An isolated recombinant vector including a nucleic acid according to one of claims 1 to 4.

6. An antisense nucleic acid or ribozyme binding to a nucleic acid, in particular RNA, according to one of claims 1 to 3.

7. A substance having a binding site for a protein or peptide coded by a nucleic acid according to one of claims 1 to 4, selected from the group consisting of
a) de-activated enzymes catalysing the glycolysis,
b) inactive proteins or peptides,
c) aptamers.

8. A substance according to claim 7 in the form of a kinase-inactive form of M2-PK.

9. A substance according to claim 8, wherein the kinase-inactive form is generated by a mutation in the region of the ADP binding site and/or the ATP binding site, in particular selected from the group consisting of “M2-PK K366M, R119C, T340M, Q377K, K161N, K165M and several of these mutations”, or selected from one or more mutations according to corresponding, however from M2-PK differing amino acids according to M1-PK.

10. The use of a substance according to one of claims 6 to 9 for blocking the co-operation between a protein kinase of the mitogenic signalling cascade and an enzyme catalysing the glycolysis, in particular of the A-raf/M2-PK co-operation.

11. The use of a substance according to one of claims 6 to 9 for the production of a pharmaceutical preparation for treating cancer diseases.

12. The use of a nucleic acid according to one of claims 1 to 3 or of a protein or peptide coded thereby in a screening method for the determination of an enzyme catalysing the glycolysis and co-operating with a protein kinase of the mitogenic signalling cascade.

13. The use of a nucleic acid according to one of claims 1 to 3 or of a protein or peptide coded thereby in a screening method for the detection of a substance binding to a protein kinase of the mitogenic signalling cascade, not however catalysing the glycolysis.
